(19) United States
(12) Patent Application Publication
(10) Pub. No.: US 2023/0190133 A1
(43) Pub. Date: Jun. 22, 2023

(54) GENERATING AN INDICATOR OF CHRONIC OBSTRUCTIVE PULMONARY DISEASE

(71) Applicant: KONINKLIJKE PHILIPS N.V., EINDHOVEN (NL)

(72) Inventors: Samer Bou Jawde, Cambridge, MA (US); Pascal de Graaf, Eindhoven (NL); Harold Johannes Antonius Brans, Eindhoven (NL); Kiran Hamilton J. Dellimore, Eindhoven (NL)

(21) Appl. No.: 17/981,588

(22) Filed: Nov. 7, 2022

Related U.S. Application Data
(60) Provisional application No. 63/290,722, filed on Dec. 17, 2021.

(51) Int. Cl. A61B 5/097; A61M 16/00; A61B 5/00; A61B 5/083; A61B 5/087

(52) U.S. Cl. CPC A61B 5/097; A61M 16/0003; A61B 5/6803; A61B 5/7282; A61B 5/0836; A61B 5/087; A61B 2560/0431

(57) ABSTRACT

Provided are concepts for generating an indicator of chronic obstructive pulmonary disease (COPD) in a subject. In particular, data from a plurality of sensors, including a pressure sensor, an airflow sensor, and a CO2 concentration sensor is captured from the breath of the subject received by a mouthpiece. The sensor data is then utilised by a processing unit to generate an indicator of COPD in the subject. By utilising airway pressure, airway flow rate and expiratory CO2 concentration data, an accurate indicator of the presence and/or stage of COPD may be obtained.

### CROSS-REFERENCE TO RELATED APPLICATIONS

This patent application claims the priority benefit under 35 U.S.C. § 119(e) of U.S. Provisional Application No. 63/290,722, filed on Dec. 17, 2021, the contents of which are herein incorporated by reference.

### FIELD OF THE INVENTION

The present invention relates to the field of lung disease and particularly to the generation of an indicator of chronic obstructive pulmonary disease.

### BACKGROUND OF THE INVENTION

Chronic obstructive pulmonary disease (COPD) is a type of progressive lung disease that is the 3rd leading cause of death worldwide. Indeed, the Global Burden of Disease Study reported a prevalence of 251 million cases of COPD around the world in 2016. Globally, it is estimated that 3.17 million deaths were caused by the disease in 2015.

COPD is characterized by airflow limitation caused by inflammation of the airways and/or the permanent enlargement of air spaces, with the main symptoms including shortness of breath and a cough. At its early stage, its slow progression makes diagnosis difficult, and even if diagnosed, patients can only reduce lung function deterioration but not eliminate it. Typically, COPD progressively worsens, with everyday activities such as walking or dressing becoming difficult.

Furthermore, the best treatment strategy (e.g. medication, oxygen therapy, etc.) depends on the stage of COPD. Thus, continuous and proper assessment to determine the correct therapy is required. In other words, early diagnosis followed by continuous and reliable monitoring of a subject's condition are key to providing the best treatment at the right moment, prolonging lung function, alleviating disease burdens, and improving living comfort.

### SUMMARY OF THE INVENTION

The invention is defined by the claims.

According to an aspect of the invention, there is provided a portable system for generating an indicator of chronic obstructive pulmonary disease (COPD) in a subject. The system comprises a mouthpiece device for receiving breath of the subject; a pressure sensor configured to measure airway pressure of the received breath of the subject; an airflow sensor configured to measure airway flow rate of the received breath of the subject; a CO2 concentration sensor configured to measure expiratory CO2 concentration of the received breath of the subject; and a processing unit configured to process the measured airway pressure, airway flow rate, and expiratory CO2 concentration in order to generate an indicator of COPD in the subject.

Provided are concepts for generating an indicator of chronic obstructive pulmonary disease in a subject. In particular, data from a plurality of sensors, including a pressure sensor, an airflow sensor, and a CO2 concentration sensor is captured from the breath of the subject received by a mouthpiece. The sensor data is then utilised by a processing unit to generate an indicator of COPD in the subject. By utilising airway pressure, airway flow rate and expiratory CO2 concentration data, an accurate indicator of the presence and stage of COPD may be obtained.

By way of explanation, the indicator of COPD of the subject may be a number, alert, or other form of notification of COPD in the subject. The indicator of COPD may indicate a probability of the presence of COPD and/or a predicted severity of COPD in the subject. In this way, a caregiver/clinician may have access to the indicator of COPD, and thus determine whether to pursue a diagnosis or an assessment of the severity of COPD (i.e. using spirometry).

Moreover, the invention may overcome existing issues with COPD monitoring devices, by providing a portable/hand-held means of providing an indicator of COPD. Thus, subjects may be monitored as they progress through the various stages of COPD, without the need for visiting a caregiver.

Furthermore, the mouthpiece device (e.g. a mask, a tube, etc.) is configured to receive breath of the subject (by the subject breathing into the mouthpiece). Various sensors are supplied with the mouthpiece, such that each of the sensors may take readings that are useful in generating an indicator of COPD. Indeed, each of the sensors may be integrated within the mouthpiece.

It has been realized that, by combining pressure, flow rate, and expiratory CO2 (partial pressure and end-tidal) measurements, a more accurate indicator of COPD may be generate/calculated. Each of these measurements may individually give clues as to the presence of COPD, but by combining each, a more thorough picture of the presence of COPD in the subject may be ascertained.

In some embodiments, the portable system may comprise a hand-held device, comprising the mouthpiece, the pressure sensor, the airflow sensor, and the CO2 concentration sensor.

In this way, the portable system may be easy to use by the subject without any specific training. The processing unit may also be provided in the hand-held device, or may be on a separate device (i.e. a smartphone, laptop etc.). In any case, the processing unit may be communicatively linked to the sensor arrangement.

In some embodiments, the portable system may further comprise a drug delivery means configured to administer medication to the subject, and the processing unit may be configured, responsive to drug delivery means administering medication to the subject, to process the measured airway pressure, airway flow rate, expiratory CO2 concentration in order to generate an updated indicator of COPD in the subject.

Accordingly, a difference in an indicator in COPD can be assessed in response to a medication. As a result, the invention enables the assessment of an effectiveness of medication in treating COPD. Accordingly, a caregiver may re-assess treatment of the subject, potentially leading to an improved subject-outcome. Indeed, the drug delivery may be a nebulizer, or any other means suitable for applying a medication or other treatment to the subject.

In some embodiments, the processing unit may be further configured to control the drug delivery means using updated drug delivery setting values based on the updated indicator of COPD in the subject.

Building on the above, the portable system may also generate updated drug delivery setting values for the drug delivery means, using the updated indicator of COPD. For example, if the updated indicator of COPD has not improved over historic indicators of COPD, then the drug delivery setting values may be updated in an attempt to improve the treatment. The setting values may refer to the drug used, the time of delivery, the dosage of the medication, the frequency of drug delivery, or any other parameter of drug delivery settings. Indeed, this may further rely on an input of a clinician or the subject themselves.

In some embodiments, the portable system may further comprise a recommendation unit configured to compare a historic indicator of COPD in the subject, and the updated indicator of COPD in the subject in order to generate a medication parameter or therapy recommendation.

Moreover, the portable system may also comprise a recommendation unit configured to provide a medication or therapy recommendation. The medication or therapy recommendation may relate to the drug used, therapy type, the time of delivery, the dosage of the medication, the frequency of drug delivery, or any other parameter related to the treatment of the subject. Thus, a caregiver may be aided with more information in order to provide the subject with the most appropriate treatment.

In some embodiments, the indicator of COPD may be a COPD value representative of a predicted stage of COPD in the subject, and the processing unit may be configured to calculate the COPD value based on at least one of the measured airway pressure, the airway flow rate, and the expiratory CO2 concentration. In this case, the portable system may further comprise an alert unit configured to notify a user responsive to the COPD value exceeding a threshold value for a predetermined length of time.

Thus, not only is the potential presence of COPD accounted for, but the potential severity of COPD is also accounted for. For example, an air flow much lower than normal may indicate COPD at a later stage than an air flow only slightly lower than normal. This prediction of stage may supply a caregiver with more information in order to supply recommend a more effective treatment strategy.

Also, by monitoring a predicted stage of COPD, this may be used to provide an alert/alarm/notification to a user or caregiver if the predicted stage of COPD remains high for too long. The threshold value may be a predetermined threshold, or may be set by the subject or caregiver.

In some embodiments, the indicator of COPD in the subject may comprise at least one of a subject effort value, a work of breathing value, a respiratory resistance value, a respiratory compliance value, a tidal volume and a respiratory rate value. The processing unit may be further configured to calculate the at least one subject effort value, work of breathing value, respiratory resistance value, respiratory compliance value, and respiratory rate value based on the measured airway pressure and airway flow rate for each subject breath received by the mouthpiece.

The sensors supplied by the invention capture data that may be used to calculate each of a subject effort value, a work of breathing value, a respiratory resistance value, a respiratory compliance value, and a respiratory rate value. Each of these parameters provides a useful indication/sign/hint at the presence and severity of COPD in the subject. This information may then be supplied to a caregiver in order to conduct further diagnosis, or to decide upon an appropriate treatment strategy.

In some embodiments, the portable system may further comprise an oxygen saturation sensor configured to measure oxygen saturation of blood of the subject, and the processing unit may be configured to process the measured airway pressure, airway flow rate, expiratory CO2 concentration and oxygen saturation in order to generate an indicator of COPD in the subject.

An oxygen saturation sensor may also provide useful data for assessing the presence of COPD, and therefore for providing an accurate indicator of COPD in the subject. In other words, by providing an oxygen saturation sensor, a blood oxygen level of the subject may also be assessed alongside the other data, facilitating a more accurate generation from an indicator of COPD in the subject.

In some embodiments, the processing unit may further comprise an exacerbation analysis unit configured to process the measured airway pressure, airway flow rate, expiratory CO2 concentration, oxygen saturation and historic exacerbation data in order to provide an exacerbation prediction value.

By way of explanation, exacerbations are a sudden worsening in a subject's condition. The importance of detecting exacerbations for diagnosing a subject has become increasingly apparent. It has also been shown that exacerbations may be predicted from airway pressure, airway flow rate, expiratory CO2 concentration, and oxygen saturation of the subject, given historic exacerbation information of the subject is known. For example, it may be known that a sudden decrease in oxygen saturation levels are an indicator of the presence of an exacerbation in the subject. Thus, the information gathered by the portable system may be used to predict exacerbations.

Indeed, the exacerbation prediction value may provide a probability of an exacerbation in the near future, or whether an exacerbation is currently occurring. This information, provided to the subject or a caregiver, may be invaluable in mitigating deterioration of the subject's condition.

In some embodiments, the historic exacerbation data may comprise subject-specific historic exacerbation data, including at least one of: feedback provided by the subject; observations provided by a clinician; and measured airway pressure, airway flow rate, oxygen saturation, and expiratory CO2 concentration corresponding to previous exacerbations.

A clinician and a subjects observations, as well of data previously gathered directly from the subject, may be gathered to determine a timeline for a subject's typical exacerbation. Thus, by gathering the above information, a more accurate exacerbation prediction value may be obtained.

In some embodiments, generating the indictor of COPD may be further based on at least one physiological attribute of the subject, and preferably wherein the at least one physiological attribute of the subject comprise at least one of: an age, a sex, a height, a weight, a BMI, present medical conditions, a medical history, an exposure to air pollution, and a smoking history.

In this way, rather than comparing the captured data (and other values derived form said data) to average/typical values (or values supplied by a caregiver), the data may be compared to typical values considering the physiological attributes of the subject. This may lead to a more accurate indicator of COPD.

In some embodiments, the mouthpiece device may be a mask covering the nose and mouth of the subject.

For the accurate measurement of airway pressure, airway flow rate, and expiratory CO2 concentration, it is important that all exhaled flow of the subject is measured. Alternatively, the subject's nostrils may be blocked.

In some embodiments, the portable system may further comprise an interface configured to output the indicator of COPD to a user.

According to a further aspect of the invention, there is provided a method for generating an indicator of chronic obstructive pulmonary disease (COPD) in a subject, the method comprising: measuring an airway pressure, an airway flowrate, and an expiratory CO2 concentration of received breath of the subject, responsive to the subject breathing into a mouthpiece device; and processing the measured airway pressure, airway flow rate, and expiratory CO2 concentration in order to generate an indicator of COPD in the subject.

According to another aspect of the invention, there is provided a computer program comprising computer program code means adapted, when said computer program is run on a computer, to implement a method for generating an indicator of chronic obstructive pulmonary disease (COPD) in a subject.

These and other aspects of the invention will be apparent from and elucidated with reference to the embodiment(s) described hereinafter.

### BRIEF DESCRIPTION OF THE DRAWINGS

For a better understanding of the invention, and to show more clearly how it may be carried into effect, reference will now be made, by way of example only, to the accompanying drawings, in which:

FIG. 1 illustrates a simplified schematic of a device for generating an indicator of COPD according to an aspect of an exemplary embodiment;

FIG. 2 shows a graph representative of typical airway pressure and airway flow as a function of time during two inhalation-exhalation cycles of a subject;

FIG. 3 shows a graph representative of typical CO2 concentration and blood oxygen saturation as a function of time during two inhalation-exhalation cycles of a subject;

FIG. 4 depicts a respiratory model and its corresponding electrical analogy;

FIG. 5 is a simplified block diagram of a system for generating an indicator of COPD in a subject according to an exemplary embodiment; and

FIG. 6 is a flow diagram of a method for generating an indicator of COPD in a subject according to another exemplary embodiment.

### DETAILED DESCRIPTION OF THE EMBODIMENTS

The invention will be described with reference to the Figures.

It should be understood that the detailed description and specific examples, while indicating exemplary embodiments of the apparatus, systems and methods, are intended for purposes of illustration only and are not intended to limit the scope of the invention. These and other features, aspects, and advantages of the apparatus, systems and methods of the present invention will become better understood from the following description, appended claims, and accompanying drawings. It should be understood that the Figures are merely schematic and are not drawn to scale. It should also be understood that the same reference numerals are used throughout the Figures to indicate the same or similar parts.

Variations to the disclosed embodiments can be understood and effected by those skilled in the art in practicing the claimed invention, from a study of the drawings, the disclosure and the appended claims. In the claims, the word “comprising” does not exclude other elements or steps, and the indefinite article “a” or “an” does not exclude a plurality. If the term “adapted to” is used in the claims or description, it is noted the term “adapted to” is intended to be equivalent to the term “configured to”.

According to proposed concepts, a number of possible solutions may be implemented separately or jointly. That is, although these possible solutions may be described below separately, two or more of these possible solutions may be implemented in one combination or another.

Embodiments of the invention aim to provide concepts for generating an indicator of chronic obstructive pulmonary disease (COPD) in a subject. In particular, data from a pressure sensor, an airway sensor and a CO2 concentration sensor is processed by a processing unit in order to generate the indicator of COPD. By utilising data from each of these sensors, an accurate indication/sign/hint of possible COPD in the subject may be determined.

Moreover, the system provided by the invention is portable, and therefore does not require the subject to enter into a clinical environment for testing. This enables the possibility of remote patient monitoring/management (RPM), which is key for fully understanding the interaction between various mechanisms of COPD. Ultimately, this may enable a more accurate treatment program to be developed by a clinician.

Indeed in some embodiments, the invention may be used to assess the effectiveness of treatments/medications on the subject, suggest a therapy transition or adjustment, and in other embodiments may enable the prediction of exacerbations. Thus, embodiments of the invention may be useful to a subject from the initial stage of early diagnosis and across the different stages of therapy while offering RPM capabilities.

Typically, COPD is measured through a spirometer, which captures the ability of a subject to fully exhale. The volume exhaled and the time required to exhale a certain volume is used to diagnose COPD, as well as identify the separate stages of COPD. Briefly, there are four recognised COPD stages: mild, moderate, severe, and very severe. These stages are based on the amount of volume a subject can exhale in 1 second (forced expiratory volume in 1 second, FEV1) in relationship to their forced vital capacity (FVC). However, only 30-50% of new cases are confirmed by this method.

However, there are a number of known problems with spirometry. Indeed, there are cases where spirometry is difficult to perform (e.g. painful expiratory manoeuvres). Further, spirometry does not provide information on the severity of the patient symptoms, nor can it be used to predict the risk of exacerbations, which have become critical in diagnosing and stratifiyng COPD patients. Spirometry also does not capture other COPD hallmarks such as hyperinflation, intrinsic positive end-expiratory pressure (iPEEP), pulmonary heterogeneity, and respiratory effort.

Moreover, while early diagnosis is important, monitoring the subject is crucial to ensure the identification of an appropriate treatment method. To elaborate, in general each COPD stage requires a different therapy. In stage 1 (mild COPD), the subject may receive short-acting bronchodilators when needed. In stage 2 (moderate COPD), long-acting bronchodilators may be added. In stage 3 (severe COPD), inhaled steroids may be given if the subject suffers from repeated exacerbations. In stage 4 (very severe COPD), the subject may require long-term oxygen therapy and/or a ventilaor.

Exacerbations, which are sudden worsening in patient condition, often lead to hospital visits, and heavily accelerate lung function deterioration. Thus, predicting exacerbations and stratifying patients on exacerbation risk may prove particularly beneficial, especially with the growth in RPM programs. To add to the complexity of stratification, many COPD subjects develop hypercapnia (increase in arterial CO2 pressure), and these subjects may benefit from transitioning to non-invasive ventilation. Therefore, the treatment prescribed and when to transition between different treatments should be personalized including the ability to predict exacerbations and capture hypercapnia

Furthermore, COPD presents differences compared to healthy subjects at several levels including respiratory mechanics and blood gases. Recently, it has been shown that volume capnography (a plot of expired CO2 concentration as a function of expired volume) may be used to detect COPD, and also differentiate the different stages of COPD. However, the capnograph results are also a function of the subject's respiratory mechanics. In addition, a subject's blood oxygen saturation level (SpO2) was shown to help in predicting exacerbations using RPM. It is also known that impaired gas exchange and hyperinflation in COPD leads to increased ventilator demand and muscle effort both during exercise and normal breathing. Thus, capturing subject respiratory effort also provides a clue as to changes in a subject's condition.

Put briefly, spirometery fails to provide insight regarding a level of COPD in the subject, and also misses out on the benefit that assessment of all the above described data provides.

Based on the above, it is clear that there exists a need for an improved means of diagnosis, monitoring, and treatment of COPD patients. In other words, the early diagnosis of COPD patients, provision of appropriate treatment across different stages of COPD, exacerbation prediction, and detecting hypercapnia are areas that require improvement.

Embodiments of the invention may be useful to a subject from the initial stage of early diagnosis and across the different stages of therapy while offering RPM capabilities. For this purpose, embodiments combine multiple and simultaneous measurements of breathing waveforms, respiratory mechanics, capnography, and pulse oximetry to more accurately assess and monitor status of the subject.

Indeed, in order to overcome the above described problems, embodiments of the invention may include the following features:

(i) The coupling, in a single hand-held device, of several sensors including a flow sensor, a pressure sensor, a CO2 concentration sensor (i.e. a capnograph or transcutaneous CO2 sensor), and a blood oxygen sensor (i.e. a pulse oximeter) that collect data simultaneously and continually for a time period;

(ii) The utilization of the collected sensor data by analysis carried out through the device's algorithm to determine several factors pertaining to the patient condition including but not limited to:
(a) Respiratory mechanics, capnography, and pulse oximetry of the subject during a session of use, and/or across several sessions (i.e. time trend analysis).
(b) A trend of the subject (e.g. stable, deteriorating, or improving) as a function of time, therapy/medication, intervention, etc.
(c) Predicting exacerbations and notifying subject/caregiver and/or therapist for early intervention.

Moreover, embodiments of the invention may provide a portable system that can be used as a spirometer. Compared to a regular hand-held spirometer, the portable system may supplement data typically captured during spirometry with data from the other sensors further aiding in a more accurate assessment and monitoring of COPD.

Other embodiments of the invention may include a drug delivery means (i.e. a nebulizer) to support drug delivery. Compared to normal nebulizers, the integration of nebulizers in the portable system of the invention may allow the direct assessment of how subjects react to the drug delivered. This enables an analysis regarding whether the administration method of the drug is efficient, whether the drug concentration is appropriate, and whether additional or different medication is needed.

In yet further embodiments, there may be provided with an interface to that may include a questionnaire to assess the subject's symptoms and exacerbations. By inputting this information, the coupling of the sensor data with these questionnaires may provide a connection between subject data and exacerbations, and thus improve their prediction and management.

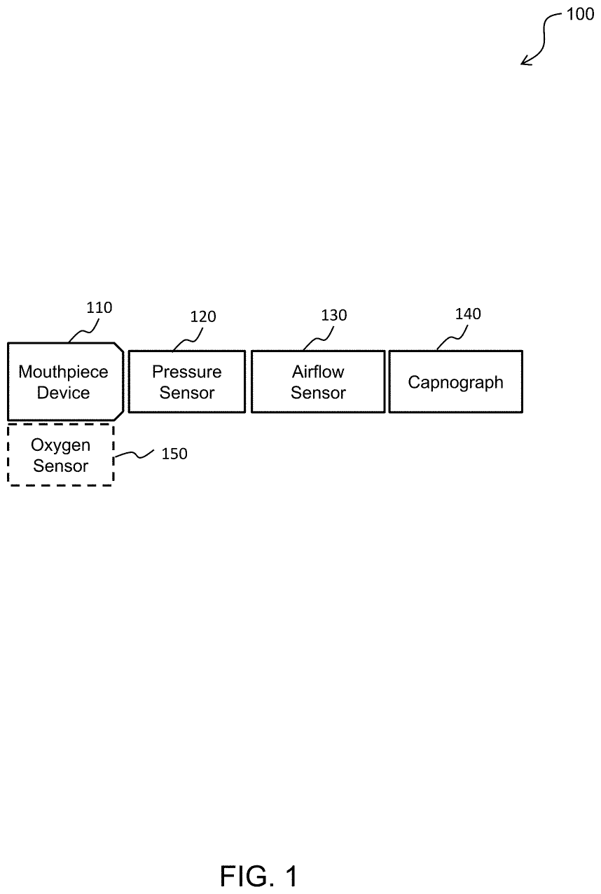

FIG. 1 illustrates a simplified schematic of a portable system 100 for generating an indicator of COPD according to an aspect of an exemplary embodiment.

Several configurations of the physical portable system 100 may be utilised. In one exemplary embodiment, the portable system 100 may externally look like a tube with a mouthpiece 110, and a CO2 concentration sensor 140 (capnograph), airflow sensor 130, and pressure sensor 120 could be installed along the tube. In this case, the pressure sensor 120 could be the first sensor just after the mouthpiece 110 to best estimate airway pressure, followed by a mainstream capnograph 140 to estimate the partial pressure of CO2 in the exhaled respiratory gas (PrCO2), and then a flow sensor 130.

Alternatively, a side stream capnograph 140 may be installed in the portable system 100 (which would then require an additional side connection from the main tube). In either case, at the end of exhalation of each breath of the subject, the end-tidal CO2 (EtCO2) is obtained.

As yet another alternative, CO2 may be measured transcutanesouly (i.e. across the depth of the skin). To be measured, a transcutaneous CO2 sensor must be in contact with the subject's skin (similar to a pulse oximeter).

Further, the mouthpiece 110 may also include a blood oxygen saturation sensor 150 (i.e. a pulse oximeter). In this case, the oxygen saturation sensor 150 may take a measurement internally within the cheek or buccal area. Alternatively, the oxygen saturation sensor 150 could be used on the nose. In this case, side connections could extend from the main body to correctly position the oxygen saturation sensor 150. This has the additional benefit to eliminate flow from the nostrils, which may be necessary to best estimate airflow of the subject (i.e. total flow should come through the mouth).

Thus, some embodiments of the invention may ensure that the mouthpiece device 110 is designed to estimate the total flow of the patient while avoiding leakage. For example, nostril clips should be used to ensure that no air flows through it. The oxygen saturation sensor 150 may be used as part of the nose clip and not physically connected to the main device 110. However, the oxygen saturation data 150 is still shared with a processing unit. Another option is to also have the device 100 with a small mask to cover both the mouth and the nose with the oxygen saturation sensor 150 on the nose, and the flow sensor 130 still across the tube which is connected to the mask. In this way, the flow measured by the flow sensor 130 is an estimate of the total flow coming from both the mouth and the nose.

Accordingly, airway pressure, airway flow, blood oxygen saturation, and CO2 concentration (partial pressure and end-tidal) are collected across time by these sensors. From this data, patient effort, work of breathing (WOB), respiratory resistance, respiratory compliance, tidal volume, and respiratory rate may be calculated for each breath, and can then be averaged across breath cycles. This data may be compared across time to detect long-term deterioration (e.g. drop in lung function) or suggest short-term therapy requirements with change in COPD stage (e.g. need for bronchodilators in stage 2 or oxygen support in stage 4).

Furthermore, the data collected may be used to estimate if an exacerbation might occur. Indeed, events of exacerbation may be recorded and related to the subject's personal data. The logging of exacerbations may be done from electronic medical records (EMR), or from the patients themselves (through an interface).

For handling the system, depending on the design it might be possible to hold the system steady simply through the mouthpiece (e.g. the subject holds tightly to the device by properly looking it in their mouth). Alternatively, if the device body is long enough the subject can simply hold it with one or two hands. Another option is to add a handle perpendicular to the body of the device.

Typically, the subject may utilize the portable system during rest by simply inhaling and exhaling through the device (e.g. every morning after waking up) to collect around 2 minutes of continuous data (airway pressure and flow, capnography, and optionally pulse oximetry) from the sensors. The data is then processed by a processing unit using the method described below. The processing unit may be integrated in the portable system itself, or in the subject's smartphone or any other similar device with connectivity to the portable system.

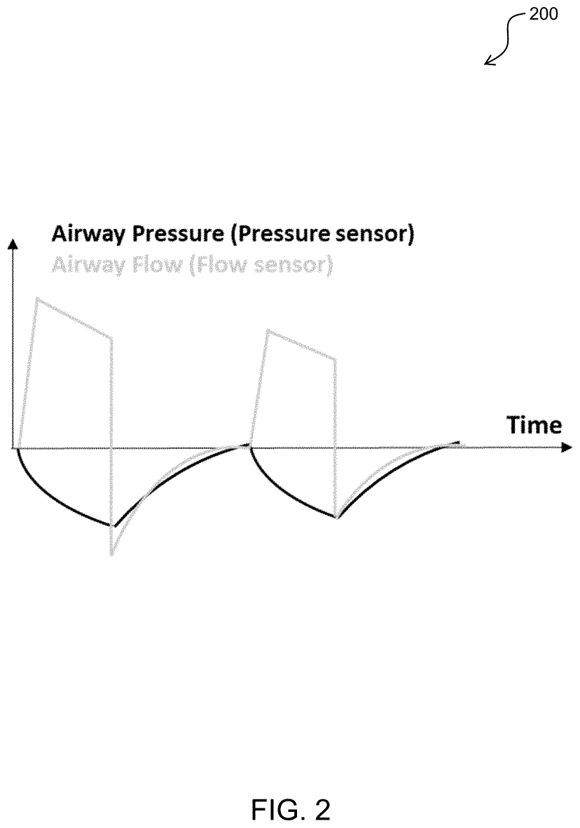

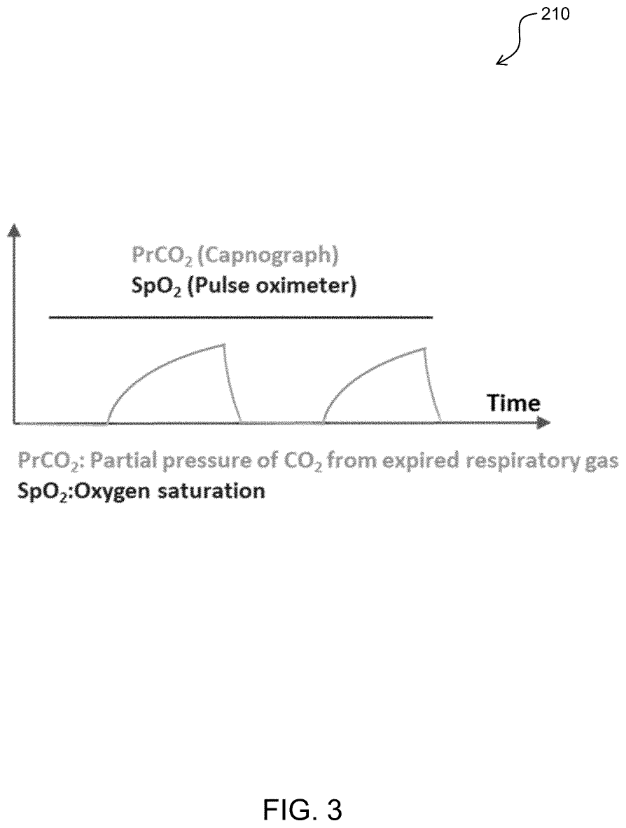

FIG. 2 presents a graph 200 representative of typical airway pressure and airway flow as a function of time during two inhalation-exhalation cycles of a subject. FIG. 3 presents a graph 210 representative of typical CO2 concentration and bloody oxygen saturation as a function of time during two inhalation-exhalation cycles of a subject. Overall, FIG. 2 and FIG. 3 represent information which may be acquired by the portable device, which may then be processed to generate the indicator of COPD in the subject.

Moving on, according to an aspect of embodiments of the invention, the indicator of COPD may be a COPD score. The COPD score could aid in the assessment of the condition of the subject, and alert clinicians for the need to transition to different therapies. The collected data may be combined into the COPD score, by first averaging the values capture for each parameter using the following equation:

$$\overline{{x}_{i}} = \frac{1}{n} {\sum }_{j}^{n} {x}_{i , j} \qquad Eq . ( 1 )$$

Where n represents the number of breaths collected during a session (e.g. 30 breaths in the morning), x_{i,j} represents a single value for a measured parameter i and for a single breath number j. Thus, for example, x_{1,2} is the measurement of SpO2 for breath number 2. An average value for a measurement across all breaths is given by x̅ᵢ:

The value of x̅ᵢ may then transposed into a score si ranging from 1 to 5 (or a different arbitrary constant), where 1 signifies that the measurement is within normal range (i.e. likely no presence of COPD) and 5 signifies the values is severely abnormal (i.e. highly likely presence of COPD). The transposition could be either set based on the average physiological ranges for the population, or may be set to be more personal (i.e. set by the subject's physician). After standardizing all parameters to the range of 1 to 5 an average score or the COPD score could be determined across all parameters:

$$COPD score = \frac{1}{N} {\sum }_{i}^{N} {s}_{i} \qquad Eq . ( 2 )$$

Where N represents the total number of parameters measured (e.g. SpO2, resistance, etc.) by the portable system.

Thus, if the COPD score is larger than 2 for more than 1 week then the subject may be provided by a recommendation from the portable system (e.g. medication not efficient). If the COPD score persists for two weeks then the subject might need to escalate treatment or transition to a different treatment, and could then visit a clinician. It could also be possible that following the two weeks the portable system itself alerts the clinician.

Moreover, the COPD score outlined here is an illustrative score and other scores could be utilized which include other types of parameters the portable system could provide (e.g. flow-volume curve . . . ). For example, one beneficial parameter that has been recently shown is the quotient between exhaled CO2 volume and the hypothetical CO2 which could be obtained from volume capnography.

Furthermore, future studies could help assess what are the best ranges and values utilized. Additionally, the parameters could be weighted depending on their importance. Finally, clinicians can always investigate the collected data from the portable system without the COPD score to assess the subject condition based on their judgement.

By way of an illustrative example, the portable system could be utilized in the following example of a subject having COPD.

The subject sets up the portable device with their physiological information (i.e. age, weight, gender, etc.). This information could be utilized to set the healthy physiological ranges based a general population, or a caregiver could provide these ranges based on prior experience/knowledge. After a period of use, the caregiver notices that the COPD score has risen to 3. The portable device indicates that the score increase was due to a slight increase in resistance compared to normal patients as well as reduced lung function. The caregiver then decides to pursue the gold standard and performs spirometry to check for COPD. Indeed, the subject is diagnosed by stage 1 COPD and is provided with the proper medication which includes high frequency chest wall oscillation since the subject also noted that he has been having a lot of mucus secretions. The subject feels better but keeps using the device for monitoring. In time, the WOB as well as the resistance increased despite the medication. The subject and their caregiver are alerted, and medication treatment is adjusted (e.g. dosage, medicine used, timing, etc.). However, as their COPD progressed, the subject suffers from several exacerbations. With the data collected by the portable system a relationship was found between the likelihood of exacerbation and the parameters (e.g. increased respiratory rate). With this knowledge, the caregiver advised the subject that on these days to avoid any air particles (e.g. close windows and use filters) and take extra doses of medication. This led to a drop of exacerbation and reduced the rate of lung function deterioration (i.e. compliance and resistance increased slower). As the disease though further progressed, the subject was still getting exhausted with chest pain. This also coincided with drop of SpO2 captured by the device which showed up in an elevated COPD score. The caregiver then suggests oxygen therapy which reduces the COPD score as SpO2 returns to normal level. After a long period of time the subject's EtCO2 levels start to rise even though they feel completely normal. The caregiver is also alerted and monitors the situation. The subject's EtCO2 levels indicated by the device are still increasing (captured by the COPD score) and then the caregiver tests and discovers that the subject has hypercapnia. Following this new diagnosis, the caregiver transitions him to non-invasive ventilation.

According to some embodiments, the portable system may include many algorithms concerned with assessing time waveforms. For example the portable system may calculate:

The peak pressure and tidal volume of every breath cycle;

(ii) The inspiratory and expiatory times of every breath cycle;

(iii) The percent variation of these parameters across breath cycles;

(iv) The estimation of intrinsic positive end-expiratory pressure (iPEEP) and volume stacking across breath cycles; and

(v) Alarms and/or alerts that are provided to the user and/or the caregiver when necessary such as: a drop in PaO2 or PaCO2 levels below a certain threshold for a given amount of time; a sudden elevation of airway resistance compared to previous time point; and inefficiency of the drug medication after several times of use.

The skilled person would understand how to calculate the above time waveforms from the data captured by the sensors of the portable system.

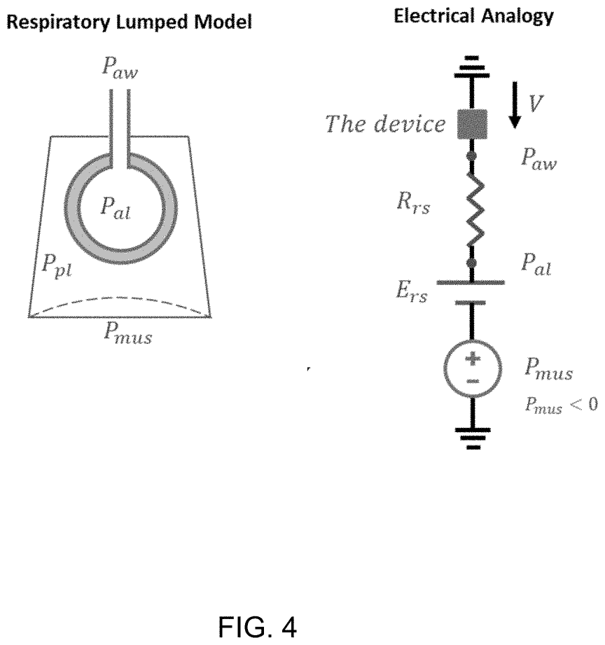

One algorithm that may be more complex is the estimation of the respiratory muscle effort by the subject. FIG. 4 shows the respiratory model and its corresponding electrical analogy used to describe the algorithm, and are provided to assist in understanding the equations below.

The model's equation of motion is given by:

P_{aw}(t)−Pₘᵤₛ(t)=Rᵣₛ\dot{V}(t)EᵣₛV(t) Eq. (3)

Where P_{aw} is the airway pressure, Pₘᵤₛ is the pressure exerted by the respiratory muscles, V is the volume added to the lung, V is the flow to the lung, Rᵣₛ is the respiratory resistance, and Eᵣₛ is the respiratory stiffness (or the inverse of compliance).

The objective of the algorithm is to estimate Rᵣₛ, Eᵣₛ, and Pₘᵤₛ given V, \dot{V}, and P_{aw}. To this end, Pₘᵤₛ may be modelled as:

$${P}_{mus} ( t ) = - {P}_{max} ( 1 - {e}^{\frac{- RR + 4 {P}_{0.1}}{10} t} ) for 0 < t \leq {T}_{i} \qquad Eq . ( 4 )$$

$${P}_{mus} ( t ) = - {P}_{max} ( 1 - {e}^{\frac{- RR + \frac{{P}_{0.1}}{2}}{10} t} ) for {T}_{i} < t \leq {T}_{i} + {T}_{e} \qquad Eq . ( 5 )$$

Where RR is the respiratory rate, P_{0.1} is the occlusion pressure or the pressure after 100 ms of a breath start, Tᵢ is the inspiratory time of the breath, and Tₑ is the expiratory time of the breath. Pₘₐₓ is the maximum pressure (a positive value) generated by the patient effort and is a function of P_{0.1} and RR:

$${P}_{max} = \frac{{P}_{0.1}}{1 - {e}^{\frac{- 0.1 ( RR + 4 {P}_{0.1} )}{10} t}} \qquad Eq . ( 6 )$$

Thus from Eqs. 4 and 3 it is evident that Pₘᵤₛ, which is a continuous function of time, can be written as a function of two variables RR and P_{0.1}. In brief, to solve for: Rᵣₛ, Eᵣₛ, RR and P_{0.1} it is necessary to fit for 4-time independent unknowns

To relate these unknowns and use a single one-parameter equation, the equation of motion may be solved at two different time points. At the end of inspiration (t=Tᵢ), the flow is zero, the volume is the tidal volume (V_{T}) and Pₘᵤₛ is −Pₘₐₓ, thus Eq. 3 can be written as:

P_{aw}(t=Tᵢ)+Pₘₐₓ=EᵣₛV_{T} Eq. (7)

Consequently, Eᵣₛ can be written as:

$${E}_{rs} = \frac{{P}_{aw} ( t = {T}_{1} ) + {P}_{max} ( {P}_{0.1} , RR )}{{V}_{T}} \qquad Eq . ( 8 )$$

For the 2^{nd} relation, we will equate Eq. 3 at 100 ms, where Pₘᵤₛ is equal to P_{0.1} and assume the tidal volume contribution to pressure is low enough so it could be neglected. This an assumption that can be applied for simplicity and clarification. Without the assumption, the same idea still holds, and the algorithm can be applied. With the assumption the equation at t=100 ms becomes:

P_{aw}(t=0.1)+P_{0.1}=Rᵣₛ\dot{V}(t=0.1) Eq. (9)

Thus, Rᵣₛ can be written as:

$${R}_{rs} = \frac{{P}_{aw} ( t = 0.1 ) + {P}_{0.1}}{\dot{V} ( t = 0.1 )} \qquad Eq . ( 10 )$$

With Eqs. 8 and 10, Eq. 3 can be written as:

$${P}_{aw} - {P}_{mus} ( t , {P}_{0.1} , RR ) = [ \frac{{P}_{aw} ( t = 0.1 ) + {P}_{0.1}}{\dot{V} ( t = 0.1 )} ] \dot{V} ( t ) + [ \frac{{P}_{aw} ( t = T ) + {P}_{max} ( {P}_{0.1} )}{{V}_{T}} ] V ( t ) \qquad Eq . ( 11 )$$

Thus the final equation is a function of the pressure and flow at certain time points, Pₘᵤₛ parameters (RR and P_{0.1}), V_{T}, and RR. RR and V_{T} as well as Tᵢ and Tₑ can be estimated from the waveforms. Similarly, for pressure and flow at certain time points. Hence Pₘᵤₛ will be a function of P_{0.1} only. P_{0.1} can then be solved for every breath numerically. From P_{0.1} and RR, all other parameters (Rᵣₛ, Eᵣₛ, Pₘᵤₛ) are equated.

Additionally, by solving for Pₘᵤₛ, the work done by the patient or work of breathing (WOB) could be estimated from:

WOB=∫₀^{T}^{ₑ}(P_{aw}−Pₘᵤₛ)dV=∫₀^{T}^{ₑ}(P_{aw}−Pₘᵤₛ)\dot{V}dt Eq. (12)

In short, the steps for the described algorithm, applied for each breath, are as follows:

(i) Airway flow and pressure waveform data are collected;

(ii) RR, V_{T}, Tᵢ and Tₑ are estimated from theses waveforms

(iii) Eq. 11 is fitted for P_{0.1}

(iv) Rᵣₛ, Cᵣₛ, and Pₘᵤₛ are estimated

(v) WOB is calculated

Moreover, the large data input collected by the portable system could have several other advantages. For example, the portable system could also be used following exercise to see the level of strain on the subject following exercise. It could also help the subject adjust the intensity of training based on the data provided. This could be known from the subject effort and the respiratory rate.

Further, the portable system could also be used before and just after a drug therapy. For example, a subject can use the portable system for 2 minutes, administer a drug via a nebulizer, and then use the portable system again to see how the drug is performing. Drug treatment could be followed across much larger time spans, and different doses and drugs used could be optimized based on the response. Also, some embodiments may integrate the nebulizer with the portable system.

The different sensors could also be parts of the larger portable system. For instance, the system could be a sum of components. There could be a pressure sensor, a flow sensor, a pulse oximeter, and a capnograph. Each of these could function separately but also be combined in simple manner (plugging in the elements). For example, if for a specific subject capnography is of particular interest, then only that piece could be utilized. Later, the caregiver could suggest adding the pulse oximeter component. This could help reduce or divide costs across the different stages as well as have a smaller device when required.

It should also be understood that the portable system may also be used to generate an indicator for other respiratory diseases. For example, it could help differentiate between COPD patients and asthmatics which is also a well-known concern.

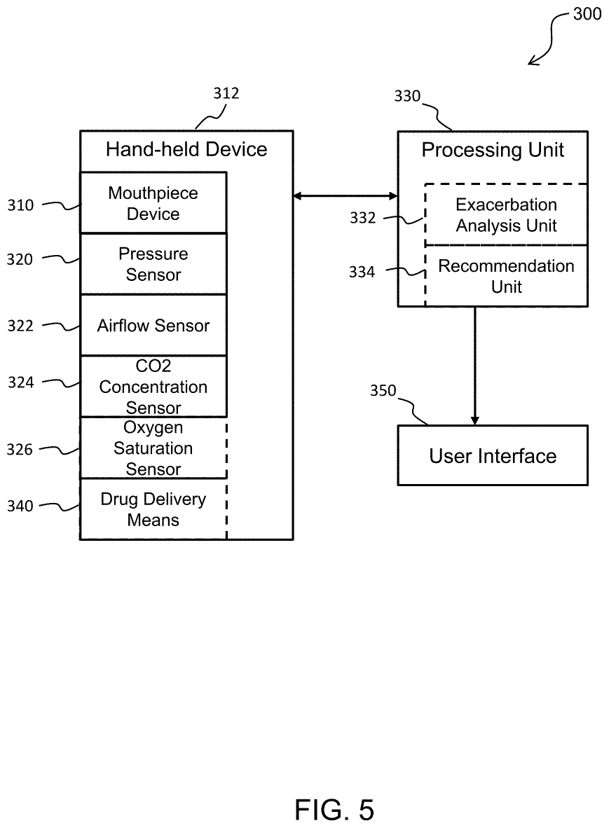

Tuning now to FIG. 5, there is presented a simplified block diagram of a portable system 300 for generating an indicator of COPD in a subject according to an exemplary embodiment. Specifically, the portable system 300 comprises a mouthpiece device 310, a pressure sensor 320, an airflow sensor 322, a CO2 concentration sensor 324, and a processing unit 330. Optionally, the system may further comprise an oxygen saturation sensor 326, a drug delivery means 340, an exacerbation analysis unit 332, a recommendation unit 334, and a user interface 350.

Firstly, the mouthpiece device 310 is configured for receiving breath of the subject. In other words, the mouthpiece device 310 is suitable for the subject to breath into, and to capture said breath. The mouthpiece device 310 may then provide the exhaled breath to the pressure sensor 320, airflow sensor 322, and CO2 concentration sensor 324.

Further, in obtaining an accurate measurement by the sensors, it is important that all of the subject's breath is captured by the mouthpiece device 310. Thus, the mouthpiece device 310 may be a mask covering the nose and mouth of the subject. Alternatively, the mouthpiece device 310 may block the nose of the subject, and only receive breath from the mouth of the subject.

Moving on to the sensors, the pressure sensor 320 is configured to measure an airway pressure of the received breath of the subject. Thus, the pressure sensor 320 may be provided closest to the mouthpiece device 310. The airflow sensor 322 is configured to measure an airway flow rate of the received breath of the subject. The airway pressure sensor 320 and airflow sensor 322 may be any sensors appropriate for measuring the described parameters, as known by the person skilled in the art.

The CO2 concentration sensor 324 is configured to measure expiratory CO2 concentration of the received breath of the subject. Indeed, the CO2 concentration sensor 324 may measure the partial pressure of CO2 in the exhaled breath of the subject (PrCO2) and/or the end-tidal CO2 in the exhaled breath of the subject (EtCO2). The CO2 concentration sensor 324 may be implemented as a mainstream capnograph, or a side-stream capnograph (which would then require an additional side connection to the mouthpiece device 310).

The processing unit 330 is configured to process the measured airway pressure, airway flow rate, and expiratory CO2 concentration captured by the above sensors in order to generate an indicator of COPD in the subject. The indicator of COPD in the subject may be a number, a word, a sensory output or any of means by which a likelihood or other pointer of COPD in the subject may be expressed.

In some embodiments, generating the indictor of COPD may be further based on at least one physiological attribute of the subject. The physiological attribute may provide some indication as to a normal value of some of the data captured by the sensors. Thus, it may be possible to compare measured values to typical values of the population for people with that physiological attribute (e.g. a measured airway pressure verses average airway pressure for people with that physiological attribute). For example, older subjects may have a lower airway pressure than an average person.

The at least one physiological attribute of the subject may comprise at least one of: an age, a sex, a height, a weight, a BMI, present medical conditions, a medical history, an exposure to air pollution, and a smoking history. Indeed, all of these factors have an impact on the lung condition of a subject, and thus the expected normal output from the sensors.

Furthermore, the indicator of COPD may be a COPD value representative of a predicted stage of COPD in the subject (i.e. mild, moderate, severe, or very severe). In this case, the processing unit 330 is configured to calculate the COPD value based on at least one of the measured airway pressure, the airway flow rate, and the expiratory CO2 concentration.

As a result, the portable system 300 may further comprise an alert unit configured to notify a user responsive to the COPD value exceeding a threshold value for a predetermined length of time.

As described in more detail above, the indicator of COPD in the subject may comprise at least one of a subject effort value, a work of breathing value, a respiratory resistance value, a respiratory compliance value, a tidal volume and a respiratory rate value. Each of these values may provide an insight into the condition of the lungs of the subject, which may be used by a clinician when performing further diagnosis.

In this case, it is the processing unit 330 that is configured to calculate the at least one subject effort value, work of breathing value, respiratory resistance value, respiratory compliance value, and respiratory rate value based on the measured airway pressure and airway flow rate for each subject breath received by the mouthpiece 310.

In addition, the portable system 300 may optionally comprise the oxygen saturation sensor 326. The oxygen saturation sensor 326 is configured to measure oxygen saturation of blood of the subject (SpO2). In this case, the processing unit 330 may be configured to process the measured airway pressure, airway flow rate, expiratory CO2 concentration and oxygen saturation in order to generate an indicator of COPD in the subject.

Additionally, the portable system 300 may comprise the drug delivery means 340. The drug delivery means 440 is configured to administer medication to the subject (e.g. via a nebulizer). In this way, the portable system 300 may provide the subject with medication, and the portable system 300 may be aware of the medication/treatment administered to the subject. In this case, the processing unit 330 may be configured, responsive to drug delivery means 340 administering medication to the subject, to process the measured airway pressure, airway flow rate, and expiratory CO2 concentration in order to generate an updated indicator of COPD in the subject.

When the drug delivery means 340 are provided, the processing unit 330 may be further configured to control the drug delivery means 340 using updated drug delivery setting values based on the updated indicator of COPD in the subject.

Alternatively, or in addition, when the drug delivery means 340 are provided, the portable system 300 may further comprise a recommendation unit 334 configured to compare a historic indicator of COPD in the subject, and the updated indicator of COPD in the subject in order to generate a medication parameter recommendation.

In some embodiments of the invention, the portable system 300 may comprise a hand-held device 312, including (integrate with) the mouthpiece device 310, the pressure sensor 320, the airflow sensor 322, and the CO2 concentration sensor 324. In this way, the means of gathering information for the generation of an indicator of COPD may be supplied by one simple-to-use means. The hand-held device 312 may also comprise the oxygen saturation sensor 326 and drug delivery means 340 in the case that they are supplied. In addition, the processing unit 330 and user interface 350 may also be provided on (integrated with) the hand-held device 312, or may be provided on a separate device.

The processing unit 330 may further comprise the exacerbation analysis unit 332 configured to process the measured airway pressure, airway flow rate, expiratory CO2 concentration, oxygen saturation and historic exacerbation data in order to provide an exacerbation prediction value. In some embodiments, the historic exacerbation data comprises subject-specific historic exacerbation data, including at least one of: feedback provided by the subject; observations provided by a clinician; and measured airway pressure, airway flow rate, oxygen saturation, and expiratory CO2 concentration corresponding to previous exacerbations.

Finally, the portable system 300 may further comprise the interface 350 configured to output the indicator of COPD to a user. The user may be a caregiver, or may be the subject. The user interface 350 may be integrated with the processing unit 330 and hand-held device 312 (i.e. a screen on the hand-held device), or may be provided separately (i.e. on a smartphone).

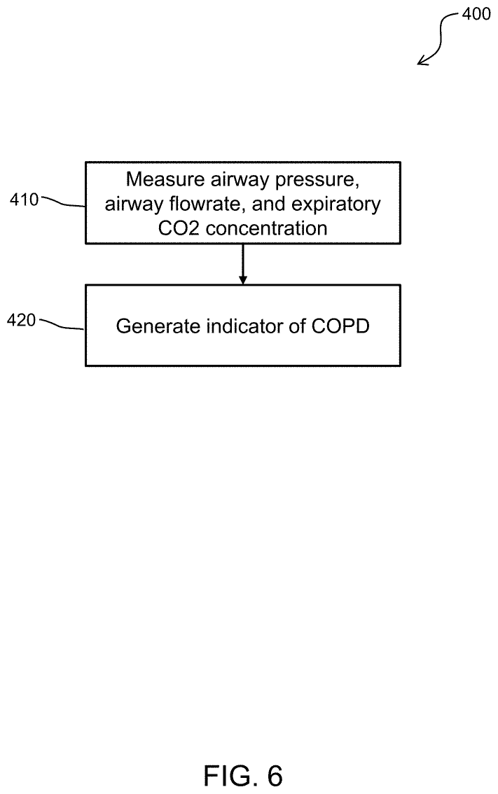

FIG. 6 is a flow diagram of a method for generating an indicator of COPD in a subject according to another exemplary embodiment.

At step 410, an airway pressure, an airway flowrate, and an expiratory CO2 concentration of received (exhaled) breath of the subject are measured. This step is performed responsive to the subject breathing into a mouthpiece device.

In this way, parameter values which may indicate the presence of COPD in the subject are acquired.

At step 420, the measured airway pressure, airway flow rate, and expiratory CO2 concentration are processed in order to generate the indicator of COPD in the subject. In other words, the measured data is leveraged to determine an indicator (likelihood of COPD).

Accordingly, an indicator of COPD is acquired, that may be utilised by a caregiver or other clinician in performing a diagnosis, or determining an appropriate treatment strategy for the subject.

A single processor or other unit may fulfil the functions of several items recited in the claims.

A computer program may be stored/distributed on a suitable medium, such as an optical storage medium or a solid-state medium supplied together with or as part of other hardware, but may also be distributed in other forms, such as via the Internet or other wired or wireless telecommunication systems.

The mere fact that certain measures are recited in mutually different dependent claims does not indicate that a combination of these measures cannot be used to advantage.

Any reference signs in the claims should not be construed as limiting the scope.

1. A portable system for generating an indicator of chronic obstructive pulmonary disease, COPD, in a subject, the system comprising:
a mouthpiece device for receiving breath of the subject;
a pressure sensor configured to measure airway pressure of the received breath of the subject;
an airflow sensor configured to measure airway flow rate of the received breath of the subject;
a CO2 concentration sensor configured to measure expiratory CO2 concentration of the received breath of the subject; and
a processing unit configured to process the measured airway pressure, airway flow rate, and expiratory CO2 concentration in order to generate an indicator of COPD in the subject.

2. The portable system of claim 1, wherein the portable system comprises a hand-held device, and wherein the hand-held device comprises the mouthpiece, the pressure sensor, the airflow sensor, and the CO2 concentration sensor.

3. The portable system of claim 1, wherein the portable system further comprises a drug delivery means configured to administer medication to the subject, and wherein the processing unit is configured, responsive to drug delivery means administering medication to the subject, to process the measured airway pressure, airway flow rate, expiratory CO2 concentration in order to generate an updated indicator of COPD in the subject.

4. The portable system of claim 3, wherein the processing unit is further configured to control the drug delivery means using updated drug delivery setting values based on the updated indicator of COPD in the subject.

5. The portable system of claim 3, wherein the processing unit further comprises a recommendation unit configured to compare a historic indicator of COPD in the subject, the updated indicator of COPD in the subject, and an indicator of COPD in a population in order to generate a medication or therapy parameter recommendation.

6. The portable system of claim 1, wherein the indicator of COPD is a COPD value representative of a predicted stage of COPD in the subject, and the processing unit is configured to calculate the COPD value based on at least one of the measured airway pressure, the airway flow rate, and the expiratory CO2 concentration, and
wherein the portable system further comprises an alert unit configured to notify a user responsive to the COPD value exceeding a threshold value for a predetermined length of time.

7. The portable system of claim 1, wherein the indicator of COPD in the subject comprises at least one of a subject effort value, a work of breathing value, a respiratory resistance value, a respiratory compliance value, and a respiratory rate value,
and wherein the processing unit is further configured to calculate the at least one subject effort value, work of breathing value, respiratory resistance value, respiratory compliance value, tidal volume, and respiratory rate value based on the measured airway pressure and airway flow rate for each subject breath received by the mouthpiece.

8. The portable system of claim 1, further comprising an oxygen saturation sensor configured to measure oxygen saturation of blood of the subject, and
wherein the processing unit is configured to process the measured airway pressure, airway flow rate, expiratory CO2 concentration and oxygen saturation in order to generate an indicator of COPD in the subject.

9. The portable system of claim 1, wherein the processing unit further comprises an exacerbation analysis unit configured to process the measured airway pressure, airway flow rate, expiratory CO2 concentration, and historic exacerbation data in order to provide an exacerbation prediction value.

10. The portable system of claim 9, wherein the historic exacerbation data comprises subject-specific historic exacerbation data, including at least one of: feedback provided by the subject; observations provided by a clinician; and measured airway pressure, airway flow rate, oxygen saturation, and expiratory CO2 concentration corresponding to previous exacerbations.

11. The portable system of claim 10, wherein generating the indictor of COPD is further based on at least one physiological attribute of the subject, and preferably wherein the at least one physiological attribute of the subject comprise at least one of: an age, a sex, a height, a weight, a BMI, present medical conditions, a medical history, an exposure to air pollution, and a smoking history.

12. The portable system of claim 1, wherein the mouthpiece device is a mask covering the nose and mouth of the subject.

13. The portable system of claim 1, further comprising an interface configured to output the indicator of COPD to a user.

14. A method for generating an indicator of chronic obstructive pulmonary disease, COPD, in a subject, the method comprising:
measuring an airway pressure, an airway flowrate, and an expiratory CO2 concentration of received breath of the subject, responsive to the subject breathing into a mouthpiece device; and
processing the measured airway pressure, airway flow rate, and expiratory CO2 concentration in order to generate an indicator of COPD in the subject.

15. A computer program comprising computer program code means adapted, when said computer program is run on a computer, to implement the method of claim 14.
